(19) United States
(12) Patent Application Publication
(10) Pub. No.: US 2004/0152696 A1
(43) Pub. Date: Aug. 5, 2004

(54) 2-substituted bicyclic benzoheterocyclic compounds and their use as sodium channel blockers

(75) Inventors: Qun Sun, Princeton, NJ (US); Donald J. Kyle, Newtown, PA (US); Samuel F. Victory, Newtown, PA (US)

Correspondence Address: STERNE, KESSLER, GOLDSTEIN & FOX PLLC, 1100 NEW YORK AVENUE, N.W., WASHINGTON, DC, 20005, US

(73) Assignee: Euro-Celtique S.A.

(21) Appl. No.: 10/628,487

(22) Filed: Jul. 29, 2003

Related U.S. Application Data
(60) Provisional application No. 60/399,702, filed on Aug. 1, 2002.

(51) Int. Cl. A61K 31/5415; A61K 31/517; C07D 279/16; C07D 239/72

(52) U.S. Cl. 514/224.2; 514/266.3; 544/50; 544/287; 514/266.31

(57) ABSTRACT

This invention relates to a method of treating disorders responsive to the blockade of sodium ion channels using novel 2-substituted bicyclic benzoheterocyclic compounds of Formula I:

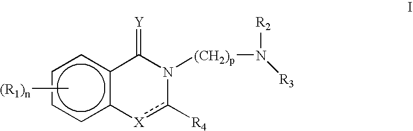

or a pharmaceutically-acceptable salt or solvate thereof, wherein X is —NH—, —N═ or —S—, Y is oxygen or sulfur, and n, p, R₁, R₂, R₃ and R₄ are defined in the specification. The invention is also directed to the use of compounds of Formula I for the treatment of neuronal damage following global or focal ischemia, for the treatment or prevention of neurodegenerative conditions such as amyotrophic lateral sclerosis (ALS), and for the treatment, prevention or amelioration of acute or chronic pain, neuropathic pain or surgical pain, as antitinnitus agents, as anticonvulsants, and as antimanic depressants, as local anesthetics, as antiarrhythmics and for the treatment or prevention of diabetic neuropathy.

This application claims the benefit under 35 U.S.C. § 119(e) of U.S. Provisional Application No. 60/399,702, filed Aug. 1, 2002, which is hereby incorporated by reference in its entirety.

### BACKGROUND OF THE INVENTION

1. Field of the Invention

This invention is in the field of medicinal chemistry. In particular, the invention relates to 2-substituted bicyclo-benzoheterocyclic compounds, and the discovery that these compounds are blockers of sodium (Na⁺) channels.

2. Related Art

Several classes of therapeutically useful drugs, including local anesthetics such as lidocaine and bupivacaine, antiarrhythmics such as propafenone and amioclarone, and anticonvulsants such as lamotrigine, phenyloin and carbamazepine, have been shown to share a common mechanism of action by blocking or modulating Na⁺ channel activity (Catterall, W. A., Trends Pharmacol. Sci. 8:57-65 (1987)). Each of these agents is believed to act by interfering with the rapid influx of Na⁺ ions.

Recently, other Na⁺ channel blockers such as BW619C89 and lifarizine have been shown to be neuroprotective in animal models of global and focal ischemia (Graham et al., J. Pharmacol. Exp. Ther. 269:854-859 (1994); Brown et al., British J. Pharmacol. 115:1425-1432 (1995)).

The neuroprotective activity of Na⁺ channel blockers is due to their effectiveness in decreasing extracellular glutamate concentration during ischemia by inhibiting the release of this excitotoxic amino acid neurotransmitter. Studies have shown that unlike glutamate receptor antagonists, Na⁺ channel blockers prevent hypoxic damage to mammalian white matter (Stys et al., J. Neurosci. 12:430-439 (1992)). Thus, they may offer advantages for treating certain types of strokes or neuronal trauma where damage to white matter tracts is prominent.

Another example of clinical use of a Na⁺ channel blocker is riluzole. This drug has been shown to prolong survival in a subset of patients with ALS (Bensimm et al., New Engl. J. Med. 330:585-591 (1994)) and has subsequently been approved by the FDA for the treatment of ALS. In addition to the above-mentioned clinical uses, carbamazepine, lidocaine and phenyloin are occasionally used to treat neuropathic pain, such as from trigeminal neurologia, diabetic neuropathy and other forms of nerve damage (Taylor and Meldrum, Trends Pharmacol. Sci. 16:309-316 (1995)), and carbamazepine and lamotrigine have been used for the treatment of manic depression (Denicott et al., J. Clin. Psychiatry 55:70-76 (1994)). Furthermore, based on a number of similarities between chronic pain and tinnitus, (Moller, A. R. Am. J. Otol. 18:577-585 (1997); Tonndorf, J Hear. Res. 28:271-275 (1987)) it has been proposed that tinnitus should be viewed as a form of chronic pain sensation (Simpson, J. J. and Davies, E. W. Tips. 20:12-18 (1999)). Indeed, lignocaine and carbamazepine have been shown to be efficacious in treating tinnitus (Majumdar, B. et al. Clin. Otolaryngol. 8:175-180 (1983); Donaldson, I. Laryngol. Otol. 95:947-951 (1981)).

It has been established that there are at least five to six sites on the voltage-sensitive Na⁺ channels which bind neurotoxins specifically (Catterall, W. A., Science 242:50-61 (1988)). Studies have further revealed that therapeutic antiarrhythmics, anticonvulsants and local anesthetics whose actions are mediated by Na⁺ channels, exert their action by interacting with the intracellular side of the Na⁺ channel and allosterically inhibiting interaction with neurotoxin receptor site 2 (Catterall, W. A., Ann. Rev. Pharmacol. Toxicol. 10:15-43 (1980)).

A need exists in the art for novel compounds that are potent blockers of sodium channels, and are therefore useful for treating a variety of central nervous system conditions, including pain.

### SUMMARY OF THE INVENTION

The present invention is related to the discovery that 2-substituted bicyclic benzoheterocyclic compounds represented by Formula I act as blockers of sodium (Na⁺) channels.

One aspect of the present invention is directed to treating disorders responsive to the blockade of sodium channels in a mammal suffering from excess activity of said channels, by administering an effective amount of a compound of Formula I, which acts as a blocker of sodium channels.

A further aspect of the present invention is to provide a method for treating, preventing or ameliorating neuronal loss following global and focal ischemia; treating, preventing or ameliorating pain including acute and chronic pain, and neuropathic pain; treating, preventing or ameliorating convulsions or neurodegenerative conditions; treating, preventing or ameliorating manic depression or diabetic neuropathy; using as local anesthetics and anti-arrhythmics, and treating tinnitus by administering a compound of Formula I to a mammal in need of such treatment or use.

Additionally, the present invention is directed to novel 2-substituted bicyclic benzoheterocyclic compounds of Formula I.

Also, the present invention provides for pharmaceutical compositions useful for treating disorders responsive to the blockade of sodium ion channels, containing an effective amount of a compound of Formula I in a mixture with one or more pharmaceutically-acceptable carriers or diluents.

Additional embodiments and advantages of the invention will be set forth in part in the description that follows, and in part will be obvious from the description, or can be learned by practice of the invention. The embodiments and advantages of the invention will be realized and attained by means of the elements and combinations particularly pointed out in the appended claims.

It is to be understood that both the foregoing general description and the following detailed description are exemplary and explanatory only, and are not restrictive of the invention as claimed.

### DETAILED DESCRIPTION OF THE INVENTION

The present invention arises out of the discovery that 2-substituted bicyclic benzoheterocyclic compounds of Formula I act as blockers of Na⁺ channels. Thus, in view of this discovery, a first aspect of the present invention is directed to a method of treating disorders responsive to the blockade of sodium ion channels using novel 2-substituted bicyclic benzoheterocyclic compounds of Formula I.

The novel 2-substituted bicyclic benzoheterocyclic compounds used in the first aspect of the present invention are represented by Formula I:

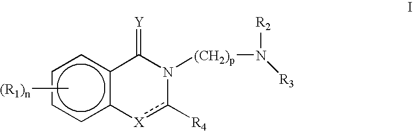

or a pharmaceutically-acceptable salt or solvate thereof, wherein:

n is an integer from zero to 3;

p is an integer from 2 to 4;

X is —N═, —NH— or —S—;

Y is oxygen or sulfur;

each occurrence of R₁ is independently selected from halogen, C₁₋₆ alkyl, C₁₋₆ alkoxy, C₁₋₆ haloalkyl, C₁₋₆ hydroxyalkyl, amino, nitro and cyano; R₂ and R₃ are independently selected from hydrogen, C₁₋₆ alkyl, C₃₋₈ cycloalkyl, C₁₋₆ haloalkyl, C₁₋₆ hydroxyalkyl and C₁₋₆ alkyloxy(C₁₋₆)alkyl; or R₂ and R₃ together with the nitrogen atom to which they are attached form a ring having 3 to 7 carbon atoms, which ring optionally contains 1 or 2 additional heteroatoms independently selected from —O—, —S— and —NR₅—, wherein each occurrence of R₅ is independently selected from hydrogen, C₁₋₆ alkyl, C₁₋₆ haloalkyl and C₁₋₆ hydroxyalkyl; and

R₄ is selected from the group consisting of:

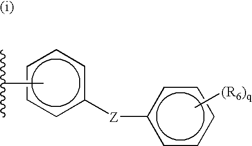

wherein:

Z is —O—, —S—, —NH—, —CH₂—, —NHCH₂—, —CH₂NH—, —OCH₂—, —CH₂O—, —SCH₂— or —CH₂S—;

each occurrence of R₆ is independently selected from halogen, C₁₋₆ alkyl, C₁₋₆ alkoxy, C₁₋₆ haloalkyl, C₁₋₆ hydroxyalkyl and C₁₋₆ alkyloxyalkyl; and

q is an integer from zero to 4;

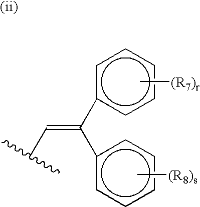

wherein:

each occurrence of R₇ and each occurrence of R₈ are independently selected from the group consisting of C₁₋₆ alkyl, C₁₋₆ haloalkyl, C₁₋₆ alkoxy, C₁₋₆ hydroxyalkyl and C₁₋₆ alkoxyalkyl;

r is an integer from zero to 4; and

s is an integer from zero to 4;

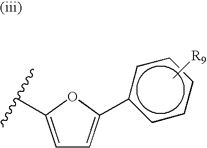

wherein:

R₉ is hydrogen, halogen or alkyl;

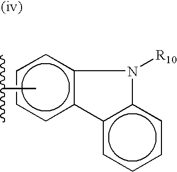

wherein:

R₁₀ is hydrogen or alkyl; and

(v) naphthyl.

The broken line in Formula I indicates that when X is —N═, the carbon atom attached to both R₄ and X is double-bonded to the X nitrogen; and when X is —NH— or —S—, the carbon atom attached to both R₄ and X is single-bonded to the X atom.

When the point of attachment of a ring to another moiety is not specified, e.g., where the connecting bond is drawn to the center of the ring, the point of attachment is at any available position on the ring, unless otherwise specified. For example, when n is 1, R₁ can be ortho, meta or para on the benzene ring relative to X; when n is 2, the two R₁ substituents can be positioned 2,3-, 2,4-, 2,5-, 3,4-, 3,5- or 4,5- on the benzene ring relative to X; and so forth.

The term “alkyl” as employed herein by itself or as part of another group refers to both straight and branched chain radicals having 1 to 10 carbon atoms, unless the chain length is otherwise specified, including, but not limited to, methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, and the like. Preferred alkyl groups include those having 1 to 6 carbon atoms.

The term “alkenyl” is used herein to mean a straight or branched chain radical of 2-10 carbon atoms, unless the chain length is otherwise specified, wherein there is at least one double bond between two of the carbon atoms in the chain, including, but not limited to, ethenyl, 1-propenyl, 2-propenyl, 2-methyl-1-propenyl, 1-butenyl, 2-butenyl, and the like. Preferably, the alkenyl chain is 2 to 8 carbon atoms in length, more preferably from 2 to 4 carbon atoms in length.

The term “alkynyl” is used herein to mean a straight or branched chain radical of 2-10 carbon atoms, unless the chain length is otherwise specified, wherein there is at least one triple bond between two of the carbon atoms in the chain, including, but not limited to, ethynyl, 1-propynyl, 2-propynyl, and the like. Preferably, the alkynyl chain is 2 to 8 carbon atoms in length, more preferably from 2 to 4 carbon atoms in length.

In all instances herein where there is an alkenyl or alkynyl moiety as a substituent group, the unsaturated linkage, i.e., the vinyl or ethenyl linkage, is preferably not directly attached to a nitrogen, oxygen or sulfur moiety.

The term “alkoxy” or “alkyloxy” refers to any of the above alkyl groups linked to an oxygen atom. Typical examples include methoxy, ethoxy, isopropyloxy, sec-butyloxy and t-butyloxy.

The term “aryl” as employed herein by itself or as part of another group means a C₆₋₁₄ mono- or polycyclic aromatic ring system. Preferably the ring system contains 6 to 10 carbon atoms. Typical examples include phenyl, naphthyl, phenanthryl, anthracyl, indenyl, azulenyl, biphenyl, biphenylenyl and fluorenyl groups. Particularly useful carbocyclic aryl groups include phenyl and naphthyl.

The term “aralkyl” or “arylalkyl” as employed herein by itself or as part of another group refers to C₁₋₆ alkyl groups as discussed above having an aryl substituent, including, but not limited to, benzyl, phenylethyl or 2-naphthylmethyl.

The term “heteroaryl” as employed herein refers to groups having 5 to 14 ring atoms; sharing 6, 10 or 14 pi electrons in a cyclic array; and containing carbon atoms and 1, 2, 3 or 4 heteroatoms independently selected from oxygen, nitrogen and sulfur. Examples of heteroaryl groups include thienyl, benzo[b]thienyl, naphtho[2,3-b]thienyl, thianthrenyl, furanyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4αH-carbazolyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl and tetrazolyl groups. Preferred heteroaryl groups are pyridyl, carbazolyl, furanyl and imidazolyl.

The term “heterocycle” as employed herein, by itself or as part of another group, refers to a saturated or partially unsaturated ring system having 5 to 14 ring atoms selected from carbon atoms and 1, 2, 3 or 4 heteroatoms independently selected from oxygen, nitrogen and sulfur. Typical examples of saturated heterocycles include pyrrolidinyl, imidazolidinyl, pyrazolidinyl, tetrahydrofuranyl, tetrahydropyranyl, piperidyl, piperazinyl, quinuclidinyl, morpholinyl and dioxacyclohexyl. Typical examples of partially unsaturated heterocycles include pyrrolinyl, imidazolinyl, pyrazolinyl, dihydropyridinyl, tetrahydropyridinyl, and dihydropyranyl. Each of these systems is optionally fused to a benzene ring.

The terms “heteroarylalkyl” or “heteroaralkyl” as employed herein both refer to a heteroaryl group attached to a C₁₋₆ alkyl group. Typical examples include 2-(3-pyridyl)ethyl, 3-(2-furyl)-n-propyl, 3-(3-thienyl)-n-propyl and 4-(1-isoquinolinyl)-n-butyl.

The term “cycloalkyl” as employed herein by itself or as part of another group refers to cycloalkyl groups containing 3 to 9 carbon atoms, unless the size is otherwise specified. Typical examples include cyclopropyl, cyclobutyl, cyclopentyl, cyclohexyl, cycloheptyl and cyclooctyl.

The term “halogen” or “halo” as employed herein by itself or as part of another group refers to chlorine, bromine, fluorine or iodine.

The term “monoalkylamine” or “monoalkylamino” as employed herein by itself or as part of another group refers to the group NH₂ wherein one hydrogen has been replaced by an alkyl group, as defined above.

The term “dialkylamine” or “dialkylamino” as employed herein by itself or as part of another group refers to the group NH2 wherein both hydrogens have been replaced by alkyl groups, as defined above.

The term “hydroxyalkyl” as employed herein refers to any of the above alkyl groups wherein one or more hydrogens thereof are replaced with one or more hydroxyl moieties.

The term “haloalkyl” as employed herein refers to any of the above alkyl groups wherein one or more hydrogens thereof are substituted by one or more halo moieties. Typical examples include fluoromethyl, difluoromethyl, trifluoromethyl, trichloroethyl, trifluoroethyl, fluoropropyl and bromobutyl.

The term “optionally substituted,” when not further defined, means optional replacement of one or more carbon-attached hydrogens with halogen, halo(C₁₋₆) alkyl, aryl, heterocycle, cycloalkyl, C₁₋₆ alkyl, C₂₋₆ alkenyl, C₂₋₆ alkynyl, aryl(C₁₋₆) alkyl, aryl(C₂₋₆) alkenyl, aryl(C₂₋₆) alkynyl, cycloalkyl(C₁₋₆) alkyl, heterocyclo(C₁₋₆ alkyl), hydroxy(C₁₋₆) alkyl, amino(C₁₋₆) alkyl, carboxy(C₁₋₆) alkyl, alkyloxy(C₁₋₆) alkyl, nitro, amino, ureido, cyano, acylamino, hydroxy, thiol, acyloxy, azido, alkyloxy, carboxy, aminocarbonyl and C₁₋₆ alkylthiol. Preferred optional substituents on a linear carbon chain include halogen, hydroxy, alkoxy, cyano, amino, nitro, aryl, heteroaryl and heterocycle. Preferred optional substituents on a carbon atom that is part of a ring system include halogen, hydroxy, alkoxy, cyano, amino, nitro, aryl, heteroaryl, heterocycle and alkyl.

Preferred values of n include zero and 1. A more preferred value of n is zero.

When n is other than zero, R₁ is preferably positioned meta or para relative to X.

Preferred values of p include 2 and 3. A more preferred value of p is 2.

Preferred Y is oxygen.

Preferred R₂ and R₃ include R₂ and R₃ that together with the nitrogen to which they are attached form a ring having 3 to 7 carbon atoms, which ring optionally contains 1 or 2 additional heteroatoms independently selected from —O—, —S— and —NR₅—, wherein R₅ is as defined above. More preferred R₂ and R₃ include R₂ and R₃ that together with the nitrogen to which they are attached form a ring having 4 or 5 carbon atoms, which ring optionally contains 1 or 2 additional heteroatoms independently selected from —O—, —S— and —NR₅—, wherein R₅ is as defined above, wherein the ring is preferably piperidyl. Preferred R₂ and R₃ also include hydrogen and C₁₋₆ alkyl.

Preferred R₄ include moieties (i) and (ii), as defined above.

When R₄ is (i), preferred Z include —O—, —S—, —OCH₂—, —CH₂O—, —SCH₂— and —CH₂S—. More preferred Z include —O—, —S—, —OCH₂— and —CH₂O—. Particularly preferred Z include —O—, —OCH₂— and —CH₂O—.

When R₄ is (i), preferred values of q include zero, 1 and 2.

When R₄ is (i), preferred R₆ include halogen, C₁₋₆ alkyl and C₁₋₆ haloalkyl. More preferred R₆ include halogen, C₁₋₄ alkyl and C₁₋₄ haloalkyl.

Useful R₄ when R₄ is (i) include 4-(4-fluorophenoxy)phenyl, 3-(3,4-dichlorophenoxy)phenyl, 3-(3-trifluoromethylphenoxy)phenyl, 3-benzyloxyphenyl and 3-(4-tert-butylphenoxy)phenyl.

When R₄ is (ii), preferred values of r include zero and 1. A more preferred value of r is zero.

When R₄ is (ii), preferred values of s include zero and 1. A more preferred value of s is zero.

When R₄ is (ii), preferred R₇ and R₈ include halogen, C₁₋₆ alkyl and C₁₋₆ haloalkyl. More preferred R₇ and R₈ include halogen, C₁₋₄ alkyl and C₁₋₄ haloalkyl.

Useful R₄ when R₄ is (ii) include 2,2-diphenylethenyl.

When R₄ is (i), the R₄ moiety is preferably attached to the bicyclic benzoheterocyclic core meta or para relative to Z.

When R₄ is (iii), preferred R₉ include hydrogen, halogen and C₁₋₆ alkyl.

When R₄ is (iv), preferred R₁₀ include hydrogen and C₁₋₆ alkyl.

Preferred compounds of Formula I include those wherein X is —NH—; Y is oxygen; R₂ and R₃ are independently selected from hydrogen, C₁₋₆ alkyl, C₁₋₆ haloalkyl, C₁₋₆ hydroxyalkyl and C₁₋₆ alkyloxy(C₁₋₆)alkyl; and n, R₁, p and R₄ are as defined above. More preferred compounds of Formula I in which X is —NH— include those wherein n is zero or 1, preferably zero; Y is oxygen; p is 2 or 3; R₂ and R₃ are independently hydrogen or C₁₋₆ alkyl; R₄ is either of moieties (i) or (ii) as defined above; and R₁ is as defined above. Particularly preferred compounds of Formula I in which X is —NH— include those wherein n is zero; Y is oxygen; p is 2; R₂ and R₃ are independently hydrogen or C₁₋₆ alkyl; and R₄ is moiety (i) listed above, wherein Z is —O—, and R₆ and q are as defined above; or R₄ is moiety (ii) as defined above.

Preferred compounds of Formula I in which X is —NH— also include those wherein Y is oxygen; R₂ and R₃ together with the nitrogen atom to which they are attached form a ring having 3 to 7 carbon atoms, which ring optionally contains 1 or 2 additional heteroatoms independently selected from —O—, —S— and —NR₅—, wherein R₅ is as defined above; and n, R₁, p and R₄ are as defined above. More preferred compounds of Formula I in which X is —NH—include those wherein n is zero or 1, preferably zero; Y is oxygen; p is 2 or 3; R₂ and R₃ together with the nitrogen to which they are attached for m a ring having 4 or 5 carbon atoms, which ring optionally contains 1 or 2 additional heteroatoms independently selected from —O—, —S— and —NR₅—, wherein R₅ is as defined above, wherein the ring is preferably piperidyl; R₄ is either of moieties (i) or (ii) as defined above; and R₁ is as defined above. Particularly preferred compounds of Formula I in which X is —NH— include those wherein n is zero; Y is oxygen; p is 2; R₂ and R₃ together with the nitrogen to which they are attached form a ring having 4 or 5 carbon atoms, which ring optionally contains 1 additional heteroatom selected from —O—, —S— and —NR₅—, wherein R₅ is as defined above, wherein the ring is preferably piperidyl; and R₄ is moiety (i) listed above, wherein Z is —O—, and R₆ and q are as defined above; or R₄ is moiety (ii) as defined above.

Preferred compounds of Formula I include those wherein X is —N═; Y is oxygen; R₂ and R₃ are independently selected from hydrogen, C₁₋₆ alkyl, C₁₋₆ haloalkyl, C₁₋₆ hydroxyalkyl and C₁₋₆ alkyloxy(C₁₋₆)alkyl; and n, R₁, p and R₄ are as defined above. More preferred compounds of Formula I in which X is —N═ include those wherein n is zero or 1, preferably zero; Y is oxygen; p is 2 or 3; R₂ and R₃ are independently hydrogen or C₁₋₆ alkyl; R₄ is either of moieties (i) or (ii) as defined above; and R₁ is as defined above. Particularly preferred compounds of Formula I in which X is —N═ include those wherein n is zero; Y is oxygen; p is 2; R₂ and R₃ are independently hydrogen or C₁₋₆ alkyl; and R₄ is moiety (i) listed above, wherein Z is —O—, and R₆ and q are as defined above; or R₄ is moiety (ii) as defined above.

Preferred compounds of Formula I in which X is —N═ also include those wherein Y is oxygen; R₂ and R₃ together with the nitrogen atom to which they are attached form a ring having 3 to 7 carbon atoms, which ring optionally contains 1 or 2 additional heteroatoms independently selected from —O—, —S— and —NR₅—, wherein R₅ is as defined above; and n, R₁, p and R₄ are as defined above. More preferred compounds of Formula I in which X is —N═ include those wherein n is zero or 1, preferably zero; Y is oxygen; p is 2 or 3; R₂ and R₃ together with the nitrogen to which they are attached form a ring having 4 or 5 carbon atoms, which ring optionally contains 1 or 2 additional heteroatoms independently selected from —O—, —S— and —NR₅—, wherein R₅ is as defined above, wherein the ring is preferably piperidyl; R₄ is either of moieties (i) or (ii) as defined above; and R₁ is as defined above. Particularly preferred compounds of Formula I in which X is —N═ include those wherein n is zero; Y is oxygen; p is 2; R₂ and R₃ together with the nitrogen to which they are attached form a ring having 4 or 5 carbon atoms, which ring optionally contains 1 additional heteroatom selected from —O—, —S— and —NR₅—, wherein R₅ is as defined above, wherein the ring is preferably piperidyl; and R₄ is moiety (i) listed above, wherein Z is —O—, and R₆ and q are as defined above; or R₄ is moiety (ii) as defined above.

Preferred compounds of Formula I include those wherein X is —S—; Y is oxygen; R₂ and R₃ are independently selected from hydrogen, C₁₋₆ alkyl, C₁₋₆ haloalkyl, C₁₋₆ hydroxyalkyl and C₁₋₆ alkyloxy(C₁₋₆)alkyl; and n, R₁, p and R₄ are as defined above. More preferred compounds of Formula I in which X is —S— include those wherein n is zero or 1, preferably zero; Y is oxygen; p is 2 or 3; R₂ and R₃ are independently hydrogen or C₁₋₆ alkyl; R₄ is either of moieties (i) or (ii) as defined above; and R₁ is as defined above. Particularly preferred compounds of Formula I in which X is —S— include those wherein n is zero; Y is oxygen; p is 2; R₂ and R₃ are independently hydrogen or C₁₋₆ alkyl; and R₄ is moiety (i) listed above, wherein Z is —O—, and R₆ and q are as defined above; or R₄ is moiety (ii) as defined above.

Preferred compounds of Formula I in which X is —S— also include those wherein Y is oxygen; R₂ and R₃ together with the nitrogen atom to which they are attached form a ring having 3 to 7 carbon atoms, which ring optionally contains 1 or 2 additional heteroatoms independently selected from —O—, —S— and —NR₅—, wherein R₅ is as defined above; and n, R₁, p and R₄ are as defined above. More preferred compounds of Formula I in which X is —S— include those wherein n is zero or 1, preferably zero; Y is oxygen; p is 2 or 3; R₂ and R₃ together with the nitrogen to which they are attached form a ring having 4 or 5 carbon atoms, which ring optionally contains 1 or 2 additional heteroatoms independently selected from —O—, —S— and —NR₅—, wherein R₅ is as defined above, wherein the ring is preferably piperidyl; R₄ is either of moieties (i) or (ii) as defined above; and R₁ is as defined above. Particularly preferred compounds of Formula I in which X is —S— include those wherein n is zero; Y is oxygen; p is 2; R₂ and R₃ together with the nitrogen to which they are attached form a ring having 4 or 5 carbon atoms, which ring optionally contains 1 additional heteroatom selected from —O—, —S— and —NR₅—, wherein R₅ is as defined above, wherein the ring is preferably piperidyl; and R₄ is moiety (i) listed above, wherein Z is —O—, and R₆ and q are as defined above; or R₄ is moiety (ii) as defined above.

Exemplary preferred compounds that can be employed in this method of the invention include, without limitation:

2-(2,2-diphenylethenyl)-3-(2-piperidin-1-ylethyl)-2,3-dihydro-1H-quinazolin-4-one;

2-[4-(4-fluorophenoxy)phenyl]-3-(2-piperidin-1-ylethyl)-2,3-dihydro-1H-quinazolin-4-one;

2-[3-(3,4-dichlorophenoxy)phenyl]-3-(2-piperidin-1-ylethyl)-2,3-dihydro-1H-quinazolin-4-one;

2-[3-(3-trifluoromethylphenoxy)phenyl]-3-(2-piperidin-1-ylethyl)-2,3-dihydro-1H-quinazolin-4-one;

2-(2,2-diphenylethenyl)-3-(2-piperidin-1-ylethyl)-benzopyrimidin-4-one;

2-[4-(4-fluorophenoxy)phenyl]-3-(2-piperidin-1-ylethyl)-benzo-pyrimidin-4-one;

2-[3-(3-trifluoromethylphenoxy)phenyl]-3-(2-piperidin-1-ylethyl)-benzopyrimidin-4-one;

2-(3-benzyloxy)phenyl-3-(2-piperidin-1-ylethyl)-2,3-dihydrobenzo-1,3-thiazin-4-one;

2-[3-(3-trifluoromethylphenoxy)phenyl]-3-(2-piperidin-1-ylethyl)-2,3-dihydrobenzo-1,3-thiazin-4-one;

2-[3-(4-tert-butyliphenoxy)phenyl]-3-(2-piperidin-1-ylethyl)-2,3-dihydrobenzo-1,3-thiazin-4-one;

2-[4-(4-fluorophenoxy)phenyl]-3-(2-piperidin-1-ylethyl)-2,3-dihydro-benzo-1,3-thiazin-4-one;

2-(2,2-diphenylethenyl)-3-(2-piperidin-1-ylethyl)-2,3-dihydrobenzo-1,3-thiazin-4-one; and

2-[3-(3,4-dichlorophenoxy)phenyl]-3-(2-piperidin-1-ylethyl)-2,3-dihydrobenzo-1,3-thiazin-4-one;

and pharmaceutically acceptable salts thereof.

Particularly preferred compounds are selected from:

2-[4-(4-fluorophenoxy)phenyl]-3-(2-piperidin-1-ylethyl)-benzo-pyrimidin-4-one;

2-[3-(3-trifluoromethylphenoxy)phenyl]-3-(2-piperidin-1-ylethyl)-benzopyrimidin-4-one;

2-(3-benzyloxy)phenyl-3-(2-piperidin-1-ylethyl)-2,3-dihydrobenzo-1,3-thiazin-4-one;

2-[3-(3-trifluoromethylphenoxy)phenyl]-3-(2-piperidin-1-ylethyl)-2,3-dihydrobenzo-1,3-thiazin-4-one;

2-[4-(4-fluorophenoxy)phenyl]-3-(2-piperidin-1-ylethyl)-2,3-dihydro-benzo-1,3-thiazin-4-one;

2-(2,2-diphenylethenyl)-3-(2-piperidin-1-ylethyl)-2,3-dihydrobenzo-1,3-thiazin-4-one; and

2-[3-(3,4-dichlorophenoxy)phenyl]-3-(2-piperidin-1-ylethyl)-2,3-dihydrobenzo-1,3-thiazin-4-one;

and pharmaceutically acceptable salts thereof.

A second aspect of the present invention is directed to novel compounds used in the method of the first aspect of the present invention, and pharmaceutical compositions thereof. Novel compounds according to this second aspect of the present invention are compounds of Formula I, as described above, provided that when R₉ is hydrogen, neither R₂ nor R₃ is hydrogen or C₁₋₆ alkyl.

In addition to administering the compound as a raw chemical, the compounds of the invention can be administered as part of a pharmaceutical preparation containing suitable pharmaceutically-acceptable carriers comprising excipients and auxiliaries that facilitate processing of the compounds into preparations that can be used pharmaceutically. Preferably, the preparations, particularly those preparations that can be administered orally and that can be used for the preferred type of administration, such as tablets, dragees and capsules, and also preparations that can be administered rectally, such as suppositories, as well as suitable solutions for administration orally or by injection, contain from about 0.01 to 99 percent, preferably from about 0.25 to 75 percent of active compound(s), together with the excipient.

Also included within the scope of the present invention are the non-toxic pharmaceutically-acceptable salts of the compounds of the present invention. Acid addition salts are formed by mixing a solution of a particular 2-substituted bicyclic benzoheterocyclic compound of Formula I, with a solution of a pharmaceutically-acceptable non-toxic acid such as, but not limited to, acetic acid, benzoic acid, carbonic acid, citric acid, dichloroacetic acid, dodecylsulfonic acid, 2-ethylsuccinic acid, fumaric acid, glubionic acid, gluconic acid, hydrobromic acid, hydrochloric acid, 3-hydroxynaphthoic acid, isethionic acid, lactic acid, lactobionic acid, levulinic acid, maleic acid, malic acid, malonic acid, methanesulfic acid, methanesulfonic acid, nitric acid, oxalic acid, phosphoric acid, propionic acid, sulfuric acid, sulfamic acid, saccharic acid, succinic acid, tartaric acid, and the like. Basic amine salts are formed by mixing a solution of the 2-substituted bicyclic benzoheterocyclic compound of the present invention with a solution of a pharmaceutically-acceptable non-toxic acid, such as those listed above, and, preferably, hydrochloric acid or carbonic acid.

The pharmaceutical compositions of the invention can be administered to any animal that can experience the beneficial effects of the compounds of the invention. Foremost among such animals are mammals, e.g., humans, dogs and cats, although the invention is not intended to be so limited.

The pharmaceutical compositions of the present invention can be administered by any means that achieve their intended purpose. For example, administration can be by parenteral, subcutaneous, intravenous, intramuscular, intraperitoneal, transdermal, or buccal routes. Alternatively, or concurrently, administration can be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings that, if desired, are resistant to gastric juices. For this purpose, concentrated saccharide solutions can be used, which optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used. Dye stuffs or pigments can be added to the tablets or dragee coatings, for example, for identification or in order to characterize combinations of active compound doses.

Other oral pharmaceutical preparations include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers can be added.

Possible pharmaceutical preparations that can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, and include, for example, sodium carboxymethyl cellulose, sorbitol, and/or dextran. Optionally, the suspension can also contain stabilizers.

A third aspect of the present invention is directed to a method of making the novel 2-substituted bicyclic benzoheterocyclic compounds of Formula I, according to the second aspect of the present invention.

The 2-substituted bicyclic benzoheterocyclic compounds of Formula I where X is —NH— are prepared by a method comprising reacting, in a first step, a 2-nitrobenzoylchloride compound with a suitable primary amine compound. The nitro moiety of the resulting product is then reduced to an amine. The amine-substituted product is then reacted with an appropriate aldehyde compound, which results in ring closure and forms the compound of Formula I where X is —NH—.

Scheme 1 depicts the method of making the compound of Formula I where X is —NH— and the primary amine is 2-piperdinylethylamine.

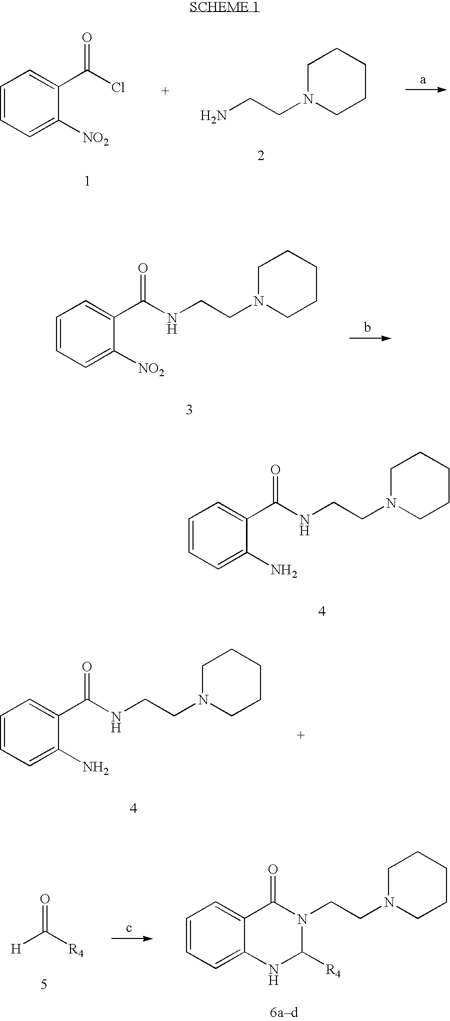

Reagents: (a) (CH₃CH₂)₃N, CH₂Cl₂; (b) H₂, Pd/C, EtOH; (c) toluene, reflux.

To obtain the compounds of Formula I where X is —N═, the process comprises reacting the final product (6a-d) of the process described above, and depicted in Scheme 1, with 2,3-dichloro-5,6-dicyano-1,4-benzoquinone (DDQ) and trichloromethane, as depicted in Scheme 2, below.

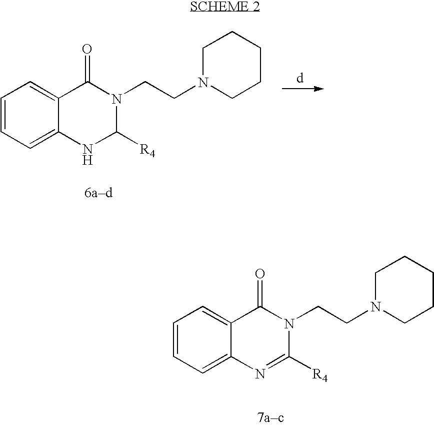

Reagents: (d) DDQ, CHCl₃.

To obtain the compounds of Formula I where X is —S—, the process comprises reacting 2-mercaptobenzoic acid (i.e., thiosalicyclic acid) with a primary amine, followed by reaction of the resulting product with an appropriate aldehyde. Scheme 3, below, shows the formation of the compound of Formula I where X is —S— and the primary amine is 2-piperidinylethylamine.

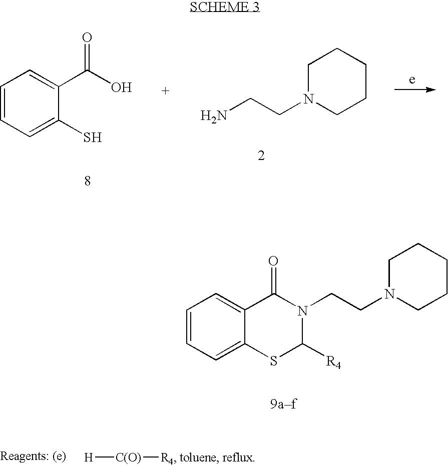

The aldehyde used in each of the above-described processes is of the formula R₄—C(O)—H, wherein R₄ can be any of the moieties (i) through (v) as described above. When R₄ is (i), (iv) or (v), the aldehyde functionality can be attached at any available position (i.e., ortho, meta or para) on the phenyl ring or the benzo portion of the fused benzene ring of R₄.

The compounds of Formula I obtained from the above-described processes are purified by flash column chromatography or silica gel chromatography.

The invention disclosed herein is meant to encompass all pharmaceutically-acceptable salts of the disclosed compounds. The pharmaceutically-acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; and amino acid salts such as arginate, asparginate, glutamate and the like.

The invention disclosed herein is also meant to encompass the in vivo metabolic products of the disclosed compounds. Such products can result, for example, from the oxidation, reduction, hydrolysis, amidation, esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabeled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein is also meant to encompass the disclosed compounds being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively.

Some of the compounds disclosed herein may contain one or more asymmetric centers and thus can give rise to enantiomers, diastereomers, and other stereoisomeric forms. The present invention is also meant to encompass all such possible forms as well as their racemic and resolved forms and mixtures thereof. When the compounds described herein contain olefinic double bonds or other centers of geometric asymmetry, and unless specified otherwise, the present invention is intended to include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached, or a sulfur atom to which three different groups are attached, where the sulfur atom and its attached groups form a sulfoxide, sulfinic ester, sulfonium salt or sulfite.

The term “enantiomer” or “enantiomeric” refers to a molecule that is nonsuperimposable on its mirror image and hence optically active such that the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule. The phrase “enantiomeric excess” refers to a mixture wherein one enantiomer is present is a greater concentration than its mirror image molecule.

The method of the first aspect of the present invention is directed to treating disorders responsive to the blockade of sodium channels in mammals suffering therefrom. Specifically, the method of the present invention utilizing the 2-substituted bicyclic benzoheterocyclic compounds of Formula I can be applied to the treatment of humans or companion animals, such as dogs and cats. Preferred 2-substituted bicyclic benzoheterocyclic compounds of Formula I for use in the method of the present invention are those as defined above.

The effectiveness of the compounds for the method of the present invention is assessed by electrophysiological assays in dissociated hippocampal neurons to determine sodium channel blocker activity. These compounds also are optionally assayed for binding to the neuronal voltage-dependent sodium channel using rat forebrain membranes and [³H]BTX-B.

Sodium channels are large transmembrane proteins that are expressed in various tissues. They are voltage-sensitive channels and are responsible for the rapid increase of Na⁺ permeability in response to depolarization associated with the action potential in many excitable cells including muscle, nerve and cardiac cells.

Another aspect of the method of the present invention is the discovery of the mechanism of action of the compounds herein described as specific Na⁺ channel blockers. Based upon the discovery of this mechanism, these compounds are contemplated to be useful in treating or preventing neuronal loss due to focal or global ischemia, and in treating or preventing neurodegenerative disorders including ALS, anxiety, and epilepsy. They are also expected to be effective in treating, preventing or ameliorating neuropathic pain, surgical pain, chronic pain and tinnitus. The compounds are also expected to be useful as antiarrhythmics, anesthetics and antimanic depressants.

The method of the present invention is directed to the use of compounds of Formula I which are blockers of voltage-sensitive sodium channels. According to the present invention, those compounds having preferred sodium channel-blocking properties exhibit an IC₅₀ of about 100 μM or less in the electrophysiological assay described herein. Preferably, the compounds of the present invention exhibit an IC₅₀ of 10 μM or less. Most preferably, the compounds of the present invention exhibit an IC₅₀ of about 1.0 μM or less. The following binding and electrophysiological assays can be used to test compounds of the present invention for their Na⁺ channel blocking activity.

In vitro Binding Assay:

The ability of compounds of the present invention to modulate either site 1 or site 2 of the Na⁺ channel was determined following the procedures fully described in Yasushi, J. Biol. Chem. 261:6149-6152 (1986) and Creveling, Mol. Pharmacol. 23:350-358 (1983), respectively. Rat forebrain membranes are used as sources of Na⁺ channel proteins. The binding assays are conducted in 130 μM choline chloride at 37° C. for 60-minute incubation with [³H] saxitoxin and [³H] batrachotoxin as radioligands for site 1 and site 2, respectively.

In Vivo Pharmacology:

The compounds of the present invention can be tested for in vivo anticonvulsant activity after i.v., p.o. or i.p. injection using a number of anticonvulsant tests in mice, including the maximum electroshock seizure test (MES). Maximum electroshock seizures are induced in male NSA mice weighing between 15-20 g and male Sprague-Dawley rats weighing between 200-225 g by application of current (50 mA, 60 pulses/sec, 0.8 msec pulse width, 1 sec duration, D.C., mice; 99 mA, 125 pulses/sec, 0.8 msec pulse width, 2 sec duration, D.C., rats) using a Ugo Basile ECT device (Model 7801). Mice are restrained by gripping the loose skin on their dorsal surface and saline-coated corneal electrodes are held lightly against the two corneae. Rats are allowed free movement on the bench top and ear-clip electrodes are used. Current is applied and animals are observed for a period of up to 30 seconds for the occurrence of a tonic hindlimb extensor response. A tonic seizure is defined as a hindlimb extension in excess of 90 degrees from the plane of the body. Results are treated in a quantal manner.

The compounds can be tested for their antinociceptive activity in the formalin model as described in Hunskaar, S., O. B. Fasmer, and K. Hole, J. Neurosci. Methods 14: 69-76 (1985). Male Swiss Webster NIH mice (20-30 g; Harlan, San Diego, Calif.) are used in all experiments. Food is withdrawn on the day of experiment. Mice are placed in Plexiglass® jars for at least 1 hour to accommodate to the environment. Following the accommodation period, mice are weighed and given either the compound of interest administered i.p. or p.o., or the appropriate volume of vehicle (10% Tween™-80). Fifteen minutes after the i.p. dosing, and 30 minutes after the p.o. dosing, mice are injected with formalin (20 μL of 5% formaldehyde solution in saline) into the dorsal surface of the right hind paw. Mice are transferred to the Plexiglass® jars and monitored for the amount of time spent licking or biting the injected paw. Periods of licking and biting are recorded in 5-minute intervals for 1 hour after the formalin injection. All experiments are done in a blinded manner during the light cycle. The early phase of the formalin response is measured as licking/biting between 0-5 minutes, and the late phase is measured from 15-50 minutes. Differences between vehicle- and drug-treated groups are analyzed by one-way analysis of variance (ANOVA). A p-value≦0.05 is considered significant. Activity in blocking the acute and second phase of formalin-induced paw-licking activity is indicative that compounds are considered to be efficacious for acute and chronic pain.

The compounds can be tested for their potential for the treatment of chronic pain (antiallodynic and antihyperalgesic activities) in the Chung model of peripheral neuropathy. Male Sprague-Dawley rats weighing between 200-225 g are anesthetized with halothane (1-3% in a mixture of 70% air and 30% oxygen) and their body temperature is controlled during anesthesia through use of a homeothermic blanket. A 2-cm dorsal midline incision is then made at the L5 and L6 level and the para-vertibral muscle groups retracted bilaterally. L5 and L6 spinal nerves are then exposed, isolated, and tightly ligated with 6-0 silk suture. A sham operation is performed exposing the contralateral L5 and L6 spinal nerves as a negative control.

Tactile Allodynia: Rats are transferred to an elevated testing cage with a wire mesh floor and allowed to acclimate for five to ten minutes. A series of Semmes-Weinstein monofilaments are applied to the plantar surface of the hindpaw to determine the animal's withdrawal threshold. The first filament used possesses a buckling weight of 9.1 g (0.96 log value) and is applied up to five times to see if it elicits a withdrawal response. If the animal has a withdrawal response then the next lightest filament in the series is applied up to five times to determine if it can elicit a response. This procedure is repeated with successively lighter filaments until there is no response, and the lightest filament that elicits a response is recorded. If the animal does not have a withdrawal response from the initial 9.1 g filament, then filaments of increased weight are successively applied until a filament elicits a response, and this filament is then recorded. For each animal, three measurements are made at every time point to produce an average withdrawal threshold determination. Tests are performed prior to and at 1, 2, 4 and 24 hours post drug administration. Tactile allodynia and mechanical hyperalgesia tests were conducted concurrently.

Mechanical Hyperalgesia: Rats are transferred to an elevated testing cage with a wire mesh floor and allowed to acclimate for five to ten minutes. A slightly blunted needle is touched to the plantar surface of the hindpaw causing a dimpling of the skin without penetrating the skin. Administration of the needle to control paws typically produces a quick flinching reaction too short to be timed with a stopwatch, and arbitrarily gives a withdrawal time of 0.5 second. The operated side paw of neuropathic animals exhibits an exaggerated withdrawal response to the blunted needle. A maximum withdrawal time of ten seconds is used as a cutoff time. Withdrawal times for both paws of the animals are measured three times at each time point with a five-minute recovery period between applications. The three measures are used to generate an average withdrawal time for each time point. Tactile allodynia and mechanical hyperalgesia tests are conducted concurrently.

The compounds can be tested for their neuroprotective activity after focal and global ischemia produced in rats or gerbils according to the procedures described in Buchan et al., Stroke, Suppl. 148-152 (1993); Sheardown et al., Eur. J. Pharmacol. 236:347-353 (1993); and Graham et al., J. Pharmacol. Exp. Therap. 276:1-4 (1996).

The compounds can be tested for their neuroprotective activity after traumatic spinal cord injury according to the procedures described in Wrathall et al., Exp. Neurology 137:119-126 (1996) and Iwasaki et al., J. Neuro Sci. 134:21-25 (1995).

Electrophysiological Assay:

An electrophysiological assay was used to measure potencies of compounds of the present invention rBIIa/beta 1 sodium channels expressed in Xenopus oocytes.

Preparation of cRNA encoding cloned rat brain type IIa (rBIIa) and beta 1 (β1): cDNA clones encoding the rat brain beta 1 subunit are cloned in house using standard methods, and mRNA are prepared by standard methods. mRNA encoding rBIIa is provided by Dr. A. Golden (UC Irvine). The mRNAs are diluted and stored at −80° C. in 1 μL aliquots until injection.

Preparation of oocytes: Mature female Xenopus laevis are anaesthetized (20-40 min) using 0.15% 3-aminobenzoic acid ethyl ester (MS-222) following established procedures (Woodward, R. M., et al., Mol. Pharmacol. 41:89-103 (1992)).

Two to six ovarian lobes are surgically removed. Oocytes at developmental stages V-VI are dissected from the ovary, wherein the oocytes are still surrounded by enveloping ovarian tissues. Oocytes are defolliculated on the day of surgery by treatment with collagenase (0.5 mg/mL Sigma Type I, or Boehringer Mannheim Type A, for 0.5-1 hr). Treated oocytes are vortexed to dislodge epithelia, washed repeatedly and stored in Barth's medium containing 88 mM NaCl, 1 mM KCl, 0.41 mM CaCl₂, 0.33 mM Ca(NO₃)₂, 0.82 mM MgSO₄, 2.4 mM NaHCO₃, 5 mM HEPES, pH 7.4 adjusted with 0.1 mg/mL gentamycin sulphate.

Micro-injection of oocytes: Defolliculated oocytes are micro-injected using a Nanoject injection system (Drummond Scientific Co., Broomall, Pa.). Injection pipettes are beveled to minimize clogging. Tip diameter of injection pipettes is 15-35 μm. Oocytes are microinjected with approximately 50 nL 1:10 ratio mixtures of cRNAs for rBIIa and beta 1 respectively.

Electrophysiology: Membrane current responses are recorded in frog Ringer solution containing 115 mM NaCl, 2 mM KCl, 1.8 mM CaCl₂, 5 mM HEPES, pH 7.4. Electrical recordings are made using a conventional two-electrode voltage clamp (Dagan TEV-200) over periods ranging between 1-7 days following injection. The recording chamber is a simple gravity fed flow-through chamber (volume 100-500 mL depending on adjustment of aspirator). Oocytes are placed in the recording chamber, impaled with electrodes and continuously perfused (5-15 mL min⁻¹) with frog Ringer's solution. The tested compounds are applied by bath perfusion.

Voltage protocols for evoking sodium channel currents: The standard holding potential for whole oocyte clamp is −120 mV. Standard current-voltage relationships are elicited by 40 ms depolarizing steps starting from −60 mV to +50 mV in 10 mV increments. Peak currents are measured as the maximum negative current after depolarizing voltage steps. The voltage from maximum current response is noted and used for the next voltage protocol.

The purpose is to find compounds that are state-dependent modifiers of neuronal sodium channels. Preferably, the compounds have a low affinity for the rested/closed state of the channel, but a high affinity for the inactivated state. The following voltage protocol is used to measure a compound's affinity for the inactivated state. Oocytes are held at a holding potential of −120 mV. At this membrane voltage, nearly all of the channels are in the closed state. Then a 4-second depolarization is made to the voltage where the maximum current is elicited. At the end of this depolarization, nearly all the channels are in the inactivated state. A 10 ms hyperpolarizing step is then made in order to remove some channels from the inactivated state. A final depolarizing test pulse is used to assay the sodium current after this prolonged depolarization (see analysis below). Sodium currents are measured at this test pulse before and after the application of the tested compound. Data is acquired using PCLAMP 8.0 software and analyzed with CLAMPFIT software (Axon instruments).

Data analysis: Apparent inhibition constants (Kᵢ values) for antagonists are determined from single point inhibition data using the following equation (a generalized form of the Cheng-Prusoff equation) (Leff, P. and Dougall, I. G., TiPS 14:110-112 (1993)):

Kᵢ=(FR/1−FR)*[drug] Eq.2

where FR is the fractional response and is defined as sodium current elicited from the final depolarizing test pulse prior to application of the drug divided by the sodium current measured in the presence of the drug, and [drug] is the concentration of the drug used.

Drugs: Drugs are initially made up at concentrations of 2-10 mM in DMSO. Dilutions are then made to generate a series of DMSO stocks over the range 0.3 μM to 10 mM, depending upon the potency of the compound. Working solutions are made by 1000- to 3000-fold dilution of stocks into Ringer. At these dilutions, DMSO alone has little or no measurable effects on membrane current responses. DMSO stocks of drugs are stored in the dark at 4° C. Ringer solutions of drugs are made up fresh each day of use.

Compositions within the scope of this invention include all compositions wherein the compounds of the present invention are contained in an amount that is effective to achieve its intended purpose. While individual needs vary, determination of optimal ranges of effective amounts of each component is within the skill of the art. Typically, the compounds can be administered to mammals, e.g., humans, orally at a dose of 0.0025 to 50 mg/kg, or an equivalent amount of the pharmaceutically-acceptable salt thereof, per day of the body weight of the mammal being treated for epilepsy, neurodegenerative diseases, anesthetic, arrhythmia, manic depression and, or chronic pain. For intramuscular injection, the dose is generally about one-half of the oral dose.

In the method of treatment or prevention of neuronal loss in global and focal ischemia, brain and spinal cord trauma, hypoxia, hypoglycemia, status epilepsy and surgery, the compound can be administrated by intravenous injection at a dose of about 0.025 to about 10 mg/kg.

The unit oral dose can comprise from about 0.01 to about 50 mg, preferably about 0.1 to about 10 mg of the compound. The unit dose can be administered one or more times daily as one or more tablets each containing from about 0.1 to about 10, conveniently about 0.25 to about 50 mg of the compound or its solvate(s).

The following non-limiting examples are illustrative of the aspects of the present invention. Other suitable modifications and adaptations of the variety of conditions and parameters normally encountered in clinical therapy and which are obvious to those skilled in the art are within the spirit and scope of the invention.

### EXAMPLE 1

### 2-Substituted 3-(2-piperidin-1-ylethyl)-2,3-dihydro-1H-quinazolin-4-one Compounds (6a-d)

To a solution of 2-nitrobenzoyl chloride 1, (3.0 g, 16.2 mmol) in dichloromethane, triethylamine (2.5 g, 24.2 mmol) and 2-piperidin-1-ylethylamine (2, 3.2 g, 24.2 mmol) were added. After 2 hours the solvent was evaporated, and compound 3 was obtained, and purified by silica gel chromatography.

To a solution of compound 3 (4.9 g, 17.6 mmol) in ethanol, 10% Pd/C (731 mg) was added. The reaction mixture was shaken under a hydrogen atmosphere at 55 psi for 1.5 hours. The resulting product was then filtered through CELITE® and concentrated. Compound 4 was obtained from the concentrate as a white solid precipitate, which was collected by filtration and washed with ethanol.

2-(2,2-diphenylethenyl)-3-(2-piperidin-1-ylethyl)-2,3-dihydro-1H-quinazolin-4-one (6a): To a solution of compound 4 (247 g, 1 mmol) in toluene, 3,3-diphenylpropenal 5, (1.0 mmol) and 4 molecular sieves were added. The solution was heated at 95° C. for 12 hours, and subsequently cooled to ambient temperature. The solvent was evaporated and the resulting compound 6a was purified by silica gel chromatography.

2-[4-(4-fluorophenoxy)phenyl]-3-(2-piperidin-1-ylethyl)-2,3-dihydro-1H-quinazolin-4-one (6b): The same procedure for compound 6a was followed, except that the aldehyde 5 used was 4-(4-fluorophenoxy)benzaldehyde.

2-[3-(3,4-dichlorophenoxy)phenyl]-3-(2-piperidin-1-ylethyl)-2,3-dihydro-1H-quinazolin-4-one (6c): The same procedure for compound 6a was followed except that the aldehyde 5 used was 3-(3,4-dichlorophenoxy)benzaldehyde.

2-[3-(3-trifluoromethylphenoxy)phenyl]-3-(2-piperidin-1-ylethyl)-2,3-dihydro-1H-quinazolin-4-one (6d): The same procedure for compound 6a was followed except that the aldehyde 5 used was (3-(3-trifluoro-methylphenoxy)benzaldehyde.

### EXAMPLE 2

### 2-Substituted 3-(2-piperidin-1-ylethyl)-benzopyrimidin-4-one Compounds (7a-c)

2-(2,2-diphenylethenyl)-3-(2-piperidin-1-ylethyl)-benzopyrimidin-4-one (7a): To a solution of compound 6a (0.05 mmol) in chloroform, 1 mL of a 0.05 M solution of DDQ in chloroform was added, and was allowed to react for 2 hours, after which the reaction mixture was placed onto a silica gel column for purification. The resulting compound 7a was removed from the eluent obtained from the purification step.

2-[4-(4-fluorophenoxy)phenyl]-3-(2-piperidin-1-ylethyl)-benzo-pyrimidin-4-one (7b): The same procedure for compound 7a was followed, except that compound 6b was used in place of compound 6a.

2-[3-(3-trifluoromethylphenoxy)phenyl]-3-(2-piperidin-1-ylethyl)-benzopyrimidin-4-one (7c): The same procedure for compound 7a was followed, except that compound 6d was used in place of compound 6a.

### EXAMPLE 3

### 2-Substituted 3-(2-piperidin-1-ylethyl)-2,3-dihydrobenzo-1,3-thiazin-4-one Compounds (9a-f)

2-(3-benzyloxy)phenyl-3-(2-piperidin-1-ylethyl)-2,3-dihydrobenzo-1,3-thiazin-4-one (9a): To a solution of 3-benzyloxybenzaldehyde 5, (1 mmol) in toluene, 2-piperidin-1-ylethylamine 2, (128 mg, 1.0 mmol), 4 molecular sieves, and 2-mercaptobenzoic acid 8, (154 mg, 1 mmol) were added. The reaction mixture was heated to 95° C., refluxed for 12 hours, and then subsequently cooled to ambient temperature. The solvent was evaporated from the reaction mixture and the retentate containing compound 9a was purified by silica gel chromatography.

2-[3-(3-trifluoromethylphenoxy)phenyl]-3-(2-piperidin-1-ylethyl)-2,3-dihydrobenzo-1,3-thiazin-4-one (9b): The same process carried out for compound 9a was used, except that the aldehyde 5 used was 3-(3-trifluoromethylphenoxy)benzaldehyde.

2-[3-(4-tert-butylphenoxy)phenyl]-3-(2-piperidin-1-ylethyl)-2,3-dihydrobenzo-1,3-thiazin-4-one (9c): The same process carried out for compound 9a was used, except that the aldehyde 5 used was 3-(4-tert-butylphenoxy)benzaldehyde.

2-[4-(4-fluorophenoxy)phenyl]-3-(2-piperidin-1-ylethyl)-2,3-dihydrobenzo-1,3-thiazin-4-one (9d): The same process carried out for compound 9a was used, except that the aldehyde 5 used was 4-(4-fluorophenoxy) benzaldehyde.

2-(2,2-diphenylethenyl)-3-(2-piperidin-1-ylethyl)-2,3-dihydro-benzo-1,3-thiazin-4-one (9e): The same process carried out for compound 9a was used, except that the aldehyde 5 used was 3,3-diphenylpropenal.

2-[3-(3,4-dichlorophenoxy)phenyl]-3-(2-piperidin-1-ylethyl)-2,3-dihydrobenzo-1,3-thiazin-4-one (9f): The same process carried out for compound 9a was used, except that the aldehyde 5 used was 3-(3,4-dichlorophenoxy)benzaldehyde.

### EXAMPLE 4

### Physical Data and Biological Activity of Compounds of the Present Invention

Physical data for compounds of the present invention are presented in Table 1. The above-described assay was used to determine the Kᵢ values for sodium channel inhibition of selected compounds of the present invention listed in Table 1. These Kᵢ values ranged from 1 to 3960 nM.

TABLE 1
Physical Properties for Compounds of the Invention

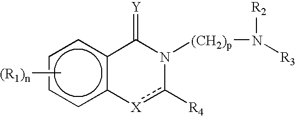

| Compound^{†} | X | R₄ | Physical Data |
|---|---|---|---|
| 6a | —NH— | 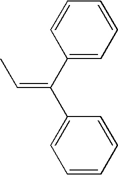 | ¹H NMR(400 MHz, CDCl₃): δ 1.32-1.47(m, 6H), 2.21(bs, 4H), 2.39(t, 2H), 2.94-3.01(m, 1H), 3.91-3.97(m, 1H), 4.39(d, 1H), 5.21(dd, 1H), 6.44(d, 1H), 6.63(d, 1H), 6.84(t, 1H), 7.15-7.47(m, 11H), 7.94(dd, 1H). MS: m/z 438.2(M + 1). |
| 6b | —NH— | 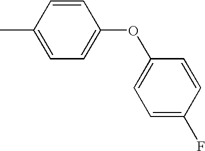 | ¹H NMR(400 MHz, CDCl₃): δ 1.39-1.57(m, 6H), 2.36-2.44(m, 5H), 2.57-2.64(m, 1H), 2.95-3.02(m, 1H), 3.95-4.02(m, 1H), 4.42(s, 1H), 5.92(d, 1H), 6.53(d, 1H), 6.83-7.35(m, 10H), 7.95(dd, 1H). MS: m/z 446.2(M + 1). |
| 6c | —NH— | 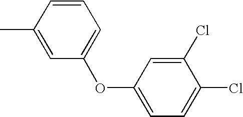 | ¹H NMR(400 MHz, CDCl₃): δ 1.39-1.58(m, 6H), 2.36-2.46(m, 5H), 2.60-2.65(m, 1H), 2.96-3.03(m, 1H), 4.03-4.08(m, 1H), 4.50(s, 1H), 5.94(d, 1H), 6.52(d, 1H), 6.82-7.51(m, 9H), 7.90(dd, 1H). MS: m/z 497.1(M + 1). |
| 6d | —NH— | 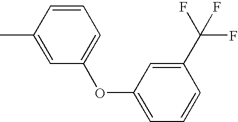 | ¹H NMR(400 MHz, CDCl₃): δ 1.40-1.58(m, 6H), 2.25-2.49(m, 5H), 2.59-2.65(m, 1H), 2.97-3.03(m, 1H), 4.02-4.09(m, 1H), 4.49(s, 1H), 5.93(d, 1H), 6.53(d, 1H), 6.77-7.41(m, 10H), 7.91(dd, 1H). MS: m/z 495.8(M + 1). |
| 7a | —N═ | 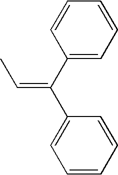 | ¹H NMR(400 MHz, CDCl₃): δ 1.37-1.56(m, 6H), 2.44(bs, 4H), 2.68(t, 2H), 4.26(t, 1H), 6.98(s, 1H), 7.16-7.63(m, 13H), 8.22(d, 1H). MS: m/z 435.8(M + 1). |
| 7b | —N═ | 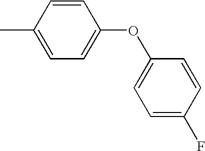 | ¹H NMR(400 MHz, CDCl₃): δ 1.35-1.49(m, 6H), 2.25(bs, 4H), 2.53(t, 2H), 4.18(t, 1H), 7.01-7.83(m, 11H), 8.31(dd, 1H). MS: m/z 444.2(M + 1). |
| 7c | —N═ | 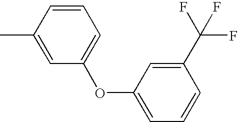 | ¹H NMR(400 MHz, CDCl₃): δ 1.36-1.50(m, 6H), 2.27(bs, 4H), 2.53(t, 2H), 4.17(t, 1H), 7.13-7.78(m, 11H), 8.31(dd, 1H). MS: m/z 494.2(M + 1). |
| 9a | —S— | 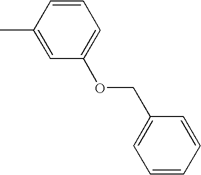 | ¹H NMR(400 MHz, CDCl₃): δ 1.41-1.60(m, 6H), 2.42(bs, 4H), 2.52-2.71(m, 2H), 3.12-3.19(m, 1H), 4.28-4.34(m, 1H), 4.96(q, 2H), 6.03(s, 1H), 6.81-7.35(m, 12H), 8.12(dd, 1H). MS: m/z 459.3(M + 1). |
| 9b | —S— | 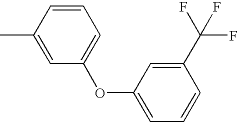 | ¹H NMR(400 MHz, CDCl₃): δ 1.41-1.57(m, 6H), 2.44(bs, 4H), 2.53-2.72(m, 2H), 3.20-3.27(m, 1H), 4.26-4.32(m, 1H), 6.07(s, 1H), 6.84-7.41(m, 11H), 8.06(dd, 1H). MS: m/z 513.3(M + 1). |
| 9c | —S— | 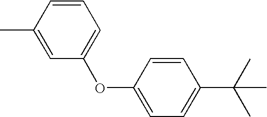 | ¹H NMR(400 MHz, CDCl₃): δ 1.33(s, 9H), 1.41-1.60(m, 6H), 2.42(bs, 4H), 2.52-2.70(m, 2H), 3.18-3.25(m, 1H), 4.23-4.30(m, 1H), 6.03(s, 1H), 6.77-7.32(m, 11H), 8.06(dd, 1H). MS: m/z 501.2(M + 1). |
| 9d | —S— | 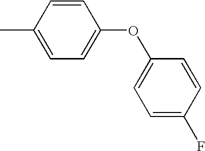 | ¹H NMR(400 MHz, CDCl₃): δ 1.41-1.62(m, 6H), 2.43(bs, 4H), 2.53-2.71(m, 2H), 3.15-3.22(m, 1H), 4.27-4.33(m, 1H), 6.04(s, 1H), 6.77-7.31(m, 11H), 8.12(dd, 1H). MS: m/z 463.2(M + 1). |
| 9e | —S— | 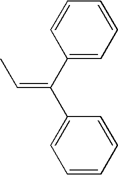 | ¹H NMR(400 MHz, CDCl₃): δ 1.25-1.42(m, 6H), 2.20(bs, 4H), 2.33-2.50(m, 2H), 3.00-3.07(m, 1H), 3.96-4.10(m, 1H), 5.17(d, 1H), 6.34(d, 1H), 7.10-7.47(m, 13H), 8.16(dd, 1H). MS: m/z 455.2(M + 1). |
| 9f | —S— | 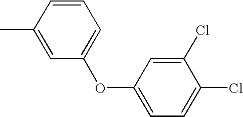 | ¹H NMR(400 MHz, CDCl₃): δ 1.42-1.61(m, 6H), 2.42(bs, 4H), 2.53-2.71(m, 2H), 3.20-3.27(m, 1H), 4.25-4.31(m, 1H), 6.06(s, 1H), 6.66-7.42(m, 10H), 8.05(dd, 1H). MS: m/z 514.1(M + 1). |

^{†}For all compounds in Table 1, n is zero, Y is oxygen, p is 2 and —NR₂R₃ is piperidyl.

### EXAMPLE 5

### Tablet Preparation

Tablets containing 25.0, 50.0, and 100.0 mg, respectively, of the compound of the invention (i.e., “active compound”) are prepared as illustrated in Table 2, below.

TABLE 2
Tablet for Doses Containing from
25-100 mg of the Active Compound

| | Amount (mg) | | |
|---|---|---|---|
| Active compound | 25.0 | 50.0 | 100.0 |
| Microcrystalline cellulose | 37.25 | 100.0 | 200.0 |
| Modified food corn starch | 37.25 | 4.25 | 8.5 |
| Magnesium stearate | 0.50 | 0.75 | 1.5 |

All of the active compound, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of active ingredient per tablet. The specific amounts of each ingredient described in Table 2 are not intended to be limiting, but are rather exemplary. The amount of active ingredient can be any amount in the range of about 25 to about 100 mg. The amounts of the remaining ingredients can thus be adjusted accordingly, as deemed necessary by those of ordinary skill in the art.

### EXAMPLE 6

### Intravenous Solution Preparation

An intravenous dosage form of the compound of the invention (i.e., “active compound”) is prepared as shown in Table 3, below.

TABLE 3
INTRAVENOUS SOLUTION FORMULATION

| | | |
|---|---|---|
| Active compound | 0.5-10.0 | mg |
| Sodium citrate | 5-50 | mg |
| Citric acid | 1-15 | mg |
| Sodium chloride | 1-8 | mg |
| Water for injection (USP) | q.s. to 1 | mL |

Utilizing the above quantities, the active compound is dissolved at room temperature in a previously-prepared solution of sodium chloride, citric acid, and sodium citrate in Water for Injection (USP, see page 1636 of United States Pharmacopeia/National Formulary for 1995, published by United States Pharmacopeial Convention, Inc., Rockville, Md. (1994)).

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All documents (e.g., scientific publications, patents and patent publications) recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A compound of Formula I: 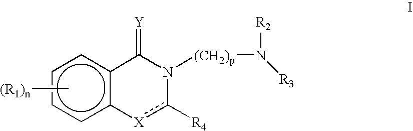
or a pharmaceutically-acceptable salt or solvate thereof, wherein:
n is an integer from zero to 3;
p is an integer from 2 to 4;
X is —N═, —NH— or —S—;
Y is oxygen or sulfur;
each occurrence of R₁ is independently selected from the group consisting of halogen, C₁₋₆ alkyl, C₁₋₆ alkoxy, C₁₋₆ haloalkyl, C₁₋₆ hydroxyalkyl, amino, nitro and cyano;
R₂ and R₃ are independently selected from the group consisting of hydrogen, C₁₋₆ alkyl, C₃₋₈ cycloalkyl, C₁₋₆ haloalkyl, C₁₋₆ hydroxyalkyl and C₁₋₆ alkyloxy(C₁₋₆)alkyl; or R₂ and R₃ together with the nitrogen atom to which they are attached form a ring having 3 to 7 carbon atoms, which ring optionally contains 1 or 2 additional heteroatoms independently selected from the group consisting of —O—, —S— and —NR₅—, wherein each occurrence of R₅ is independently selected from the group consisting of hydrogen, C₁₋₆ alkyl, C₁₋₆ haloalkyl and C₁₋₆ hydroxyalkyl; and
R₄ is selected from the group consisting of: 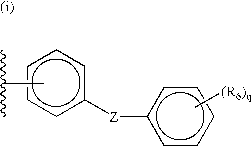
wherein:
Z is —O—, —S—, —NH—, —CH₂—, —NHCH₂—, —CH₂NH—, —OCH₂—, —CH₂O—, —SCH₂— or —CH₂S—;
each occurrence of R₆ is independently selected from the group consisting of halogen, C₁₋₆ alkyl, C₁₋₆ alkoxy, C₁₋₆ haloalkyl, C₁₋₆ hydroxyalkyl and C₁₋₆ alkyloxyalkyl; and
q is an integer from zero to 4; 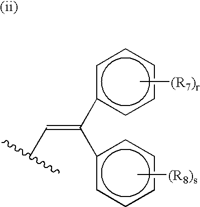
wherein:
each occurrence of R₇ and each occurrence of R₈ are independently selected from the group consisting of C₁₋₆ alkyl, C₁₋₆ haloalkyl, C₁₋₆ alkoxy, C₁₋₆ hydroxyalkyl and C₁₋₆ alkoxyalkyl;
r is an integer from zero to 4; and
s is an integer from zero to 4; 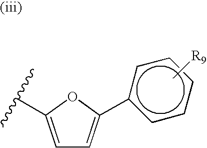
wherein:
R₉ is hydrogen, halogen or alkyl,
provided that when R₉ is hydrogen, neither R₂ nor R₃ is hydrogen or C₁₋₆ alkyl; 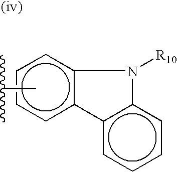
wherein:
R₁₀ is hydrogen or alkyl; and
(v) naphthyl.

2. The compound according to claim 1, wherein n is zero.

3. The compound according to claim 1, wherein p is 2.

4. The compound according to claim 1, wherein Y is oxygen.

5. The compound according to claim 1, wherein R₂ and R₃ together with the nitrogen to which they are attached form a piperidyl ring.

6. The compound according to claim 1, wherein R₄ is moiety (i).

7. The compound according to claim 6, wherein the R₄ moiety is attached to the bicyclic benzoheterocyclic core meta or para relative to Z.

8. The compound according to claim 6, wherein Z is —O—, —OCH₂— or —CH₂O—.

9. The compound according to claim 6, wherein q is zero, 1 or 2.

10. The compound according to claim 6, wherein R₆ is halogen, C₁₋₄ alkyl or C₁₋₄ haloalkyl.

11. The compound according to claim 6, wherein R₄ is 4-(4-fluoro-phenoxy)phenyl, 3-(3,4-dichlorophenoxy)phenyl, 3-(3-trifluoro-methylphenoxy)phenyl, 3-benzyloxyphenyl or 3-(4-tert-butylphenoxy)phenyl.

12. The compound according to claim 1, wherein R₄ is moiety (ii).

13. The compound according to claim 12, wherein r is zero.

14. The compound according to claim 12, wherein s is zero.

15. The compound according to claim 12, wherein R₇ and R₈ are independently selected from the group consisting of halogen, C₁₋₄ alkyl and C₁₋₄ haloalkyl.

16. The compound according to claim 12, wherein R₄ is 2,2-diphenylethenyl.

17. The compound according to claim 1, wherein:
X is —NH—;
Y is oxygen; and
R₂ and R₃ are independently selected from the group consisting of hydrogen, C₁₋₆ alkyl, C₁₋₆ haloalkyl, C-6 hydroxyalkyl and C₁₋₆ alkyloxy(C₁₋₆)alkyl, or R₂ and R₃ together with the nitrogen atom to which they are attached form a ring having 3 to 7 carbon atoms, which ring optionally contains 1 or 2 additional heteroatoms independently selected from the group consisting of —O—, —S— and —NR₅—.

18. The compound according to claim 1, wherein:
X is —NH—;
n is zero or 1;
Y is oxygen;
p is 2 or 3;
R₂ and R₃ are independently hydrogen or C₁₋₆ alkyl, or R₂ and R₃ together with the nitrogen to which they are attached form a ring having 4 or 5 carbon atoms, which ring optionally contains 1 or 2 additional heteroatoms independently selected from the group consisting of —O—, —S— and —NR₅—; and
R₄ is either of moieties (i) or (ii).

19. The compound according to claim 1, wherein:
X is —NH—;
n is zero;
Y is oxygen;
p is 2;
R₂ and R₃ together with the nitrogen to which they are attached form a ring having 4 or 5 carbon atoms, which ring optionally contains 1 additional heteroatom selected from the group consisting of —O—, —S— and —NR₅—; and
R₄ is moiety (i) wherein Z is —O—, or R₄ is moiety (ii).

20. The compound according to claim 1, wherein:
X is —N═;
Y is oxygen; and
R₂ and R₃ are independently selected from the group consisting of hydrogen, C₁₋₆ alkyl, C₁₋₆ haloalkyl, C₁₋₆ hydroxyalkyl and C₁₋₆ alkyloxy(C₁₋₆)alkyl, or R₂ and R₃ together with the nitrogen atom to which they are attached form a ring having 3 to 7 carbon atoms, which ring optionally contains 1 or 2 additional heteroatoms independently selected from the group consisting of —O—, —S— and —NR₅—.

21. The compound according to claim 1, wherein:
X is —N═;
n is zero or 1;
Y is oxygen;
p is 2 or 3;
R₂ and R₃ are independently hydrogen or C₁₋₆ alkyl, or R₂ and R₃ together with the nitrogen to which they are attached form a ring having 4 or 5 carbon atoms, which ring optionally contains 1 or 2 additional heteroatoms independently selected from the group consisting of —O—, —S— and —NR₅—; and
R₄ is either of moieties (i) or (ii).

22. The compound according to claim 1, wherein:
X is —N═;
n is zero;
Y is oxygen;
p is 2;
R₂ and R₃ together with the nitrogen to which they are attached form a ring having 4 or 5 carbon atoms, which ring optionally contains 1 additional heteroatom selected from the group consisting of —O—, —S— and —NR₅—; and
R₄ is moiety (i) wherein Z is —O—, or R₄ is moiety (ii).

23. The compound according to claim 1, wherein:
X is —S—;
Y is oxygen; and
R₂ and R₃ are independently selected from the group consisting of hydrogen, C₁₋₆ alkyl, C₁₋₆ haloalkyl, C₁₋₆ hydroxyalkyl and C₁₋₆ alkyloxy(C₁₋₆)alkyl, or R₂ and R₃ together with the nitrogen atom to which they are attached form a ring having 3 to 7 carbon atoms, which ring optionally contains 1 or 2 additional heteroatoms independently selected from the group consisting of —O—, —S— and —NR₅—.

24. The compound according to claim 1, wherein:
X is —S—;
n is zero or 1;
Y is oxygen;
p is 2 or 3;
R₂ and R₃ are independently hydrogen or C₁₋₆ alkyl, or R₂ and R₃ together with the nitrogen to which they are attached form a ring having 4 or 5 carbon atoms, which ring optionally contains 1 or 2 additional heteroatoms independently selected from the group consisting of —O—, —S— and —NR₅—; and
R₄ is either of moieties (i) or (ii).

25. The compound according to claim 1, wherein:
X is —S—;
n is zero;
Y is oxygen;
p is 2;
R₂ and R₃ together with the nitrogen to which they are attached form a ring having 4 or 5 carbon atoms, which ring optionally contains 1 additional heteroatom selected from the group consisting of —O—, —S— and —NR₅—; and
R₄ is moiety (i) wherein Z is —O—, or R₄ is moiety (ii).

26. A compound selected from the group consisting of
2-(2,2-diphenylethenyl)-3-(2-piperidin-1-ylethyl)-2,3-dihydro-1H-quinazolin-4-one;
2-[4-(4-fluorophenoxy)phenyl]-3-(2-piperidin-1-ylethyl)-2,3-dihydro-1H-quinazolin-4-one;
2-[3-(3,4-dichlorophenoxy)phenyl]-3-(2-piperidin-1-ylethyl)-2,3-dihydro-1H-quinazolin-4-one;
2-[3-(3-trifluoromethylphenoxy)phenyl]-3-(2-piperidin-1-ylethyl)-2,3-dihydro-1H-quinazolin-4-one;
2-(2,2-diphenylethenyl)-3-(2-piperidin-1-ylethyl)-benzopyrimidin-4-one;
2-[4-(4-fluorophenoxy)phenyl]-3-(2-piperidin-1-ylethyl)-benzo-pyrimidin-4-one;
2-[3-(3-trifluoromethylphenoxy)phenyl]-3-(2-piperidin-1-ylethyl)-benzopyrimidin-4-one;
2-(3-benzyloxy)phenyl-3-(2-piperidin-1-ylethyl)-2,3-dihydrobenzo-1,3-thiazin-4-one;
2-[3-(3-trifluoromethylphenoxy)phenyl]-3-(2-piperidin-1-ylethyl)-2,3-dihydrobenzo-1,3-thiazin-4-one;
2-[3-(4-tert-butylphenoxy)phenyl]-3-(2-piperidin-1-ylethyl)-2,3-dihydrobenzo-1,3-thiazin-4-one;
2-[4-(4-fluorophenoxy)phenyl]-3-(2-piperidin-1-ylethyl)-2,3-dihydro-benzo-1,3-thiazin-4-one;
2-(2,2-diphenylethenyl)-3-(2-piperidin-1-ylethyl)-2,3-dihydrobenzo-1,3-thiazin-4-one; and
2-[3-(3,4-dichlorophenoxy)phenyl]-3-(2-piperidin-1-ylethyl)-2,3-dihydrobenzo-1,3-thiazin-4-one;
or a pharmaceutically-acceptable salt or solvate thereof.

27. A pharmaceutical composition comprising the compound according to claim 1, or pharmaceutically-acceptable salt thereof, and a pharmaceutically-acceptable carrier or diluent.

28. A method of making the compound according to claim 1 wherein X is —NH—, said method comprising:
(a) reacting a 2-nitrobenzoylchloride or a 2-nitrothiobenzoyl-chloride with a compound of Formula II: 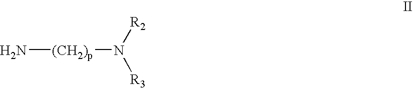
wherein p, R₂ and R₃ are as defined in claim 1;
(b) reducing the product from (a) in the presence of hydrogen;
(c) reacting the product from (b) with an aldehyde of Formula III: 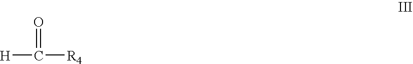
wherein R₄ is as defined in claim 1; and
(d) recovering the product obtained from (c).

29. A method of making the compound according to claim 1 wherein X is —N═, said method comprising:
(a) reacting a 2-nitrobenzoylchloride or a 2-nitrothiobenzoyl-chloride with a compound of Formula II: 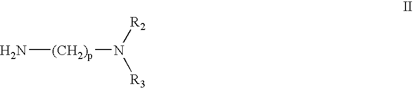
wherein p, R₂ and R₃ are as defined in claim 1;
(b) reducing the product from (a) in the presence of hydrogen;
(c) reacting the product from (b) with an aldehyde of Formula III: 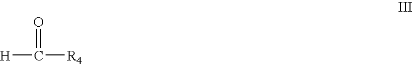
wherein R₄ is as defined in claim 1;
(d) reacting the product from (c) with 2,3-dichloro-5,6-dicyano-1,4-benzoquinone and trichloromethane; and
(e) recovering the product obtained from (d).

30. A method of making the compound according to claim 1 wherein X is —S—, said method comprising:
(a) reacting a 2-mercaptobenzoic acid or a 2-mercaptothiobenzoic acid with:
(i) a compound of Formula II: 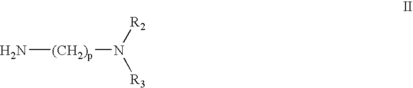
wherein p, R₂ and R₃ are as defined in claim 1; and
(ii) an aldehyde of Formula III; 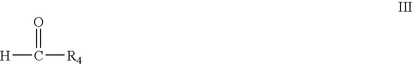
wherein R₄ is as defined in claim 1; and
(b) recovering the product obtained from (a).

31. A method of treating a mammal suffering from a disorder responsive to blockage of sodium channels, said method comprising administering to said mammal, in an amount that is effective for treating or ameliorating said disorder, a compound of Formula I: 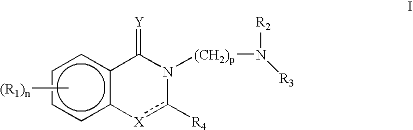
or a pharmaceutically-acceptable salt or solvate thereof, wherein:
n is an integer from zero to 3;
p is an integer from 2 to 4;
X is —N═, —NH— or —S—;
Y is oxygen or sulfur;
each occurrence of R₁ is independently selected from the group consisting of halogen, C₁₋₆ alkyl, C₁₋₆ alkoxy, C₁₋₆ haloalkyl, C₁₋₆ hydroxyalkyl, amino, nitro and cyano;
R₂ and R₃ are independently selected from the group consisting of hydrogen, C₁₋₆ alkyl, C₃₋₈ cycloalkyl, C₁₋₆ haloalkyl, C₁₋₆ hydroxyalkyl and C₁₋₆ alkyloxy(C₁₋₆)alkyl; or R₂ and R₃ together with the nitrogen atom to which they are attached form a ring having 3 to 7 carbon atoms, which ring optionally contains 1 or 2 additional heteroatoms independently selected from the group consisting of —O—, —S— and —NR₅—, wherein each occurrence of R₅ is independently selected from the group consisting of hydrogen, C₁₋₆ alkyl, C₁₋₆ haloalkyl and C₁₋₆ hydroxyalkyl; and
R₄ is selected from the group consisting of: 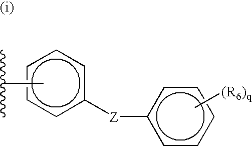
wherein:
Z is —O—, —S—, —NH—, —CH₂—, —NHCH₂—, —CH₂NH—, —OCH₂—, —CH₂O—, —SCH₂— or —CH₂S—;
each occurrence of R₆ is independently selected from the group consisting of halogen, C₁₋₆ alkyl, C₁₋₆ alkoxy, C₁₋₆ haloalkyl, C₁₋₆ hydroxyalkyl and C₁₋₆ alkyloxyalkyl; and
q is an integer from zero to 4; 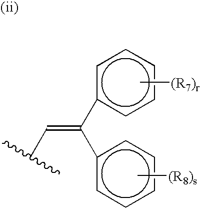
wherein:
each occurrence of R₇ and each occurrence of R₈ are independently selected from the group consisting of C₁₋₆ alkyl, C₁₋₆ haloalkyl, C₁₋₆ alkoxy, C₁₋₆ hydroxyalkyl and C₁₋₆ alkoxyalkyl;
r is an integer from zero to 4; and
s is an integer from zero to 4; 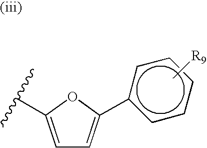
wherein:
R₉ is hydrogen, halogen or alkyl; 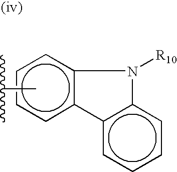
wherein:
R₁₀ is hydrogen or alkyl; and
(v) naphthyl.

32. The method according to claim 31, wherein:
X is —NH—;
Y is oxygen; and
R₂ and R₃ are independently selected from the group consisting of hydrogen, C₁₋₆ alkyl, C₁₋₆ haloalkyl, C₁₋₆ hydroxyalkyl and C₁₋₆ alkyloxy(C₁₋₆)alkyl, or R₂ and R₃ together with the nitrogen atom to which they are attached form a ring having 3 to 7 carbon atoms, which ring optionally contains 1 or 2 additional heteroatoms independently selected from the group consisting of —O—, —S— and —NR₅—.

33. The method according to claim 31, wherein:
X is —NH—;
n is zero or 1;
Y is oxygen;
p is 2 or 3;
R₂ and R₃ are independently hydrogen or C₁₋₆ alkyl, or R₂ and R₃ together with the nitrogen to which they are attached form a ring having 4 or 5 carbon atoms, which ring optionally contains 1 or 2 additional heteroatoms independently selected from the group consisting of —O—, —S— and —NR₅—; and
R₄ is either of moieties (i) or (ii).

34. The method according to claim 31, wherein:
X is —NH—;
n is zero;
Y is oxygen;
p is 2;
R₂ and R₃ together with the nitrogen to which they are attached form a ring having 4 or 5 carbon atoms, which ring optionally contains 1 additional heteroatom selected from the group consisting of —O—, —S— and —NR₅—; and
R₄ is moiety (i) wherein Z is —O—, or R₄ is moiety (ii).

35. The method according to claim 31, wherein:
X is —N═;
Y is oxygen; and
R₂ and R₃ are independently selected from the group consisting of hydrogen, C₁₋₆ alkyl, C₁₋₆ haloalkyl, C₁₋₆ hydroxyalkyl and C₁₋₆ alkyloxy(C₁₋₆)alkyl, or R₂ and R₃ together with the nitrogen atom to which they are attached form a ring having 3 to 7 carbon atoms, which ring optionally contains 1 or 2 additional heteroatoms independently selected from the group consisting of —O—, —S— and —NR₅—.

36. The method according to claim 31, wherein:
X is —N═;
n is zero or 1;
Y is oxygen;
p is 2 or 3;
R₂ and R₃ are independently hydrogen or C₁₋₆ alkyl, or R₂ and R₃ together with the nitrogen to which they are attached form a ring having 4 or 5 carbon atoms, which ring optionally contains 1 or 2 additional heteroatoms independently selected from the group consisting of —O—, —S— and —NR₅—; and
R₄ is either of moieties (i) or (ii).

37. The method according to claim 31, wherein:
X is —N═;
n is zero;
Y is oxygen;
p is 2;
R₂ and R₃ together with the nitrogen to which they are attached form a ring having 4 or 5 carbon atoms, which ring optionally contains 1 additional heteroatom selected from the group consisting of —O—, —S— and —NR₅—; and
R₄ is moiety (i) wherein Z is —O—, or R₄ is moiety (ii).

38. The method according to claim 31, wherein:
X is —S—;
Y is oxygen; and
R₂ and R₃ are independently selected from the group consisting of hydrogen, C₁₋₆ alkyl, C₁₋₆ haloalkyl, C₁₋₆ hydroxyalkyl and C₁₋₆ alkyloxy(C₁₋₆)alkyl, or R₂ and R₃ together with the nitrogen atom to which they are attached form a ring having 3 to 7 carbon atoms, which ring optionally contains 1 or 2 additional heteroatoms independently selected from the group consisting of —O—, —S— and —NR₅—.

39. The method according to claim 31, wherein:
X is —S—;
n is zero or 1;
Y is oxygen;
p is 2 or 3;
R₂ and R₃ are independently hydrogen or C₁₋₆ alkyl, or R₂ and R₃ together with the nitrogen to which they are attached form a ring having 4 or 5 carbon atoms, which ring optionally contains 1 or 2 additional heteroatoms independently selected from the group consisting of —O—, —S— and —NR₅—; and
R₄ is either of moieties (i) or (ii).

40. The method according to claim 31, wherein:
X is —S—;
n is zero;
Y is oxygen;
p is 2;
R₂ and R₃ together with the nitrogen to which they are attached form a ring having 4 or 5 carbon atoms, which ring optionally contains 1 additional heteroatom selected from the group consisting of —O—, —S— and —NR₅—; and
R₄ is moiety (i) wherein Z is —O—, or R₄ is moiety (ii).

41. The method according to claim 31, wherein said disorder is selected from the group consisting of: neuronal damage, acute or chronic pain, neuropathic pain, surgical pain, convulsions, a neurodegenerative condition, manic depression and diabetic neuropathy.

42. The method according to claim 31, wherein said disorder is acute or chronic pain.

43. The method according to claim 31, wherein said disorder is neuropathic pain.

44. The method according to claim 31, wherein said disorder is surgical pain.

45. The method according to claim 31, wherein said disorder is neuronal damage caused by focal or global ischemia.

46. The method according to claim 31, wherein said disorder is a neurodegenerative condition.

47. The method according to claim 46, wherein said neurodegenerative condition is amyotrophic lateral sclerosis (ALS).

48. The method according to claim 31, wherein said compound functions as an antitinnitus agent, an anticonvulsant, an antiarrhythmic, a local anesthetic or an antimanic depressant.

49. The method according to claim 31, wherein said mammal is a human, dog or cat.

50. The method according to claim 31, wherein said mammal is a human.
